(19) United States
(12) Patent Application Publication
(10) Pub. No.: US 2007/0299386 A1
(43) Pub. Date: Dec. 27, 2007

(54) Delivery of an ocular agent using iontophoresis

(75) Inventor: Gholam A. Peyman, Sun City, AZ (US)

Correspondence Address: WOOD, HERRON & EVANS, LLP, 2700 CAREW TOWER, 441 VINE STREET, CINCINNATI, OH, 45202, US

(73) Assignee: MINU, L.L.C., Pittsboro, NC (US)

(21) Appl. No.: 11/462,499

(22) Filed: Aug. 4, 2006

Related U.S. Application Data
(60) Provisional application No. 60/805,638, filed on Jun. 23, 2006.

(51) Int. Cl. A61N 1/30

(52) U.S. Cl. 604/20

(57) ABSTRACT

A method and apparatus for delivering an agent to structures of the eye using iontophoresis applied through the eyelid of a patient. A drug is introduced into the eye. A first electrode is in electrical communication with the eyelid and a second electrode is positioned in relation to the first electrode. The electrodes are energized to generate a current between the electrodes and through the eye that facilitates the selective dissemination of the agent throughout the eye. An apparatus for such iontophoresis administration includes a housing having an inner surface adapted to be in electrical communication with the eyelid. The first electrode is positioned in the housing and in electrical communication with at least a portion of the inner surface. The housing may also include a reservoir for holding an agent for introduction into the eye through the eyelid.

This application claims priority to provisional patent application Ser. No. 60/805,638 filed on Jun. 23, 2006, the disclosure of which is expressly incorporated by reference herein in its entirety.

### BACKGROUND

The treatment of ocular diseases in mammals, including humans and non-humans alike, often require that drugs or other agents be delivered to the eye in a therapeutic dose. Such diseases may occur in the choroid, retina, crystalline lens, optic nerve as well as other ocular structures. One treatment methodology is to deliver an ocular agent to these structures via local drug administration, as opposed to systemic drug administration. This permits agents to be delivered directly to a site requiring evaluation and/or therapy. Because of the localization, there is less of a concern for release or dissemination of the agent beyond the site of delivery. In many instances, however, local drug administration to the eye has heretofore not been easily accomplished. Thus, localized drug administration often requires rather invasive procedures to gain access to the various ocular structures being treated. This may entail inserting a conduit, such as a fine gauge needle, into the eye or forming an incision for positioning of a device, such as a drug depot, within the eye. Consequently, such treatment typically requires a visit to a hospital or doctor's office where trained health care professionals (physicians, nurses, etc.) can perform the necessary, relatively more invasive procedures to achieve local drug administration for the treatment of ocular disease.

Other treatment methodologies are desirable.

### BRIEF DESCRIPTION OF THE DRAWINGS

FIG. 1 is a perspective view of a device for delivering and/or disseminating an agent throughout the eye in accordance with an embodiment of the invention.

FIG. 2 is a cross-sectional view of the mammalian eye illustrating the device shown in FIG. 1.

FIG. 3 is an enlarged cross-sectional view of the device shown in FIG. 1.

FIG. 4 is a cross-sectional view of the eye similar to that shown in FIG. 2 illustrating an alternate embodiment in accordance with the invention.

### DETAILED DESCRIPTION

A device and method for delivering an agent to the eye in a less invasive manner is disclosed. In one embodiment, a method for ocular drug delivery includes delivering the drug by electromotive drug administration, known as iontophoresis, through the eyelid. In particular, the method provides a device that is placed over the closed eyelid and includes a first electrode (anode and/or cathode) that is in electrical communication with the surface of the eyelid. A second electrode (the other of the anode or cathode) is spaced relative to the first electrode and strategically positioned inside or outside the body so as to direct the agents in a preferred direction and within certain regions of the eye for which treatment is desired. In one embodiment, the device itself may include a reservoir for holding the one or more agents to be delivered to the eye. In such a case, the agents are capable of being transported through the closed eyelid and into the eye by iontophoresis. In another embodiment, one or more agents may be introduced into the eye through other means. For example, an agent may be topically applied to the eye, such as with eye drops, creams, emulsions, etc. In another example, a reservoir or agent depot may be positioned in the eye containing one or more agents. In any of these cases, once the agent is introduced in the eye, the device may be positioned over the eyelid and activated so as to facilitate dissemination of the agent throughout the eye using iontophoresis.

As those of ordinary skill in the art will recognize, a wide range of agents may be used with the inventive method and device for the treatment of a wide range of ocular pathologies. Pathologies may affect one or more ocular structures as shown in FIG. 2 subsequently described. A wide range of diseases may be treated including, but not limited to, immunogenic, vascular, degenerative, genetic diseases, malignancies, and diseases of any ocular structures, such as the uvea, cornea, conjuntiva, sclera, choroid, retina, lens (e.g., cataracts), optic nerve, mibomian gland, aqueous, vitreous, etc. By way of non-limiting example, the agent may include at least one of the following: a macrolide and/or mycophenolic acid, an antimicrobial agent (other antibiotics, antifungals, antivirals, etc.), anti-inflammatory agents (e.g., steroids, NSAIDs), anti-proliferative agents (e.g., anti-VEGF), hormones, cytokines, growth factors, antibodies, immune modulators, vectors for gene therapy (e.g., viral or plasmid vectors), oligonucleotides (e.g., RNA duplexes, DNA duplexes, RNAi, aptamers, antisense oligonucleotide, immunostimulatory or immunoinhibitory oligos, etc.), enzymes, enzyme inhibitors, immune modulators, etc. The agent may be in a liquid or semi-liquid form, a suspension, an emulsion, etc. Any of the above agents may be formulated as microspheres, microvesicles, microcapsules, liposomes, nanoparticles or nanocrystals of pharmaceutically active compounds, and/or nanoscale dispersions, encapsulations, and emulsions (e.g., to limit or prevent aggregation of reaggregation or crystals, to incorporate a stabilizer, etc.). The agents may be lipophilic, hydrophilic, or amphiphilic. The agents may be combined with albumin or another non-toxic solvent to form nanoparticles in a solvent-free formulation of a toxic drug. The agents may be formulated as sugar-derived nanocompounds that may shield proteins and small molecules from rapid breakdown. The agents may be rendered more soluble in a nanocrystal formulation by decreasing drug particle size and hence increasing the surface area thereby leading to an increase in dissolution. These techniques are known to one skilled in the art as disclosed in, for example, U.S. Pat. Nos. 6,822,086; 6,753,006; 6,749,868; 6,592,903; 6,537,579; 6,528,067; 6,506,405; 6,375,986; 6,096,331; 5,916,596; 5,863,990; 5,811,510; 5,665,382; 5,560,933; 5,498,421; 5,439,686; and 5,362,478; and U.S. patent application Ser. Nos. 10/106,117; 60/147,919; and 08/421,766, each of which is expressly incorporated by reference herein in its entirety.

Agents that inhibit angiogenesis include but are not limited to bevacivumab, ranibizuman, TNP470, integrin av antagonists, 2-methoxyestradiol, paclitaxel, P38 mitogen activated protein kinase inhibitors, anti-VEGF siRNA, and sunitinib maleate, geldanamycin . They may inhibit synovitis, uveitis, iritis, retinal vasculitis, optic nerve neuritis, papillitis, retinitis proliferance in diabetes, etc.

Anti-inflammatory agents include, but are not limited to, the following: colchicine; a steroid such as triamcinolone (Aristocort®; Kenalog®), anecortave acetate (Alcon), betamethasone (Celestone®), budesonide cortisone, dexamethasone (Decadron-LA®; Decadron® phosphate; Maxidex® and Tobradex® (Alcon)), hydrocortisone methylprednisolone (Depo-Medrol®, Solu-Medrol®), prednisolone (prednisolone acetate, e.g., Pred Forte® (Allergan), Econopred and Econopred Plus® (Alcon), AK-Tate® (Akorn), Pred Mild® (Allergan), prednisone sodium phosphate (Inflamase Mild and Inflamase Forte® (Ciba), Metreton® (Schering), AK-Pred® (Akorn)), fluorometholone (fluorometholone acetate (Flarex® (Alcon), Eflone®), fluorometholone alcohol (FML® and FML-Mild®, (Allergan), FluorOP®), rimexolone (Vexol® (Alcon)), medrysone alcohol (HMS® (Allergan)), lotoprednol etabonate (Lotemax® and Alrex® (Bausch & Lomb), and 11-desoxcortisol; an anti-prostaglandin such as indomethacin; ketorolac tromethamine; ((±)-5-benzoyl-2,3-dihydro-1H-pyrrolizine-1-carboxylic acid, a compound with 2-amino-2-(hydroxymethyl)-1,3-propanediol (1:1) (Acular® Allegan), Ocufen( (flurbiprofen sodium 0.03%), meclofenamate, fluorbiprofen, and the pyrrolo-pyrrole group of non-steroidal anti-inflammatory drugs; a macrolide such as sirolimus (rapamycin), pimocrolous, tacrolimus (FK506), cyclosporine (Arrestase), everolimus 40-O-(2-hydroxymethylenrapamycin), ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, lincosamide, biolimus, ABT-578 (methylrapamycin), and derivatives of rapamycin such as temsirolimus (CCI-779, Wyeth) and AP23573 (Ariad); a non-steroidal anti-inflammatory drug such as derivatives of acetic acid (e.g. diclofenac and ketorolac (Toradol®, Voltaren®, Voltaren-XR®, Cataflam®)), salicylate (e.g., aspirin, Ecotrin®), proprionic acid (e.g., ibuprofen (Advil®, Motrin®, Medipren®, Nuprin®)), acetaminophen (Tylenol®), aniline (e.g., aminophenolacetaminophen, pyrazole (e.g., phenylbutazone), N-arylanthranilic acid (fenamates) (e.g., meclofenamate), indole (e.g., indomethacin (Indocin®, Indocin-SR®)), oxicam (e.g., piroxicam (Feldene®)), pyrrol-pyrrole group (e.g., Acular®)), antiplatelet medications, choline magnesium salicylate (Trilisate®), cox-2 inhibitors (meloxicam (Mobic®)), diflunisal (Dolobid®), etodolac (Lodine®), fenoprofen (Nalfon®), flurbiprofen (Ansaid®), ketoprofen (Orudis®, Oruvail ®), meclofenamate (Meclomen®), nabumetone (Relafen®), naproxen (Naprosyn®, Naprelan®, Anaprox®, Aleve®), oxaprozin (Daypro®), phenylbutazone (Butazolidine®)), salsalate (Disalcid®, Salflex®), tolmetin (Tolectin(®), valdecoxib (Bextra®), sulindac (Clinoril®), and flurbiprofin sodium (Ocufen®), an MMP inhibitor such as doxycycline, TIMP-1, TIMP-2, TIMP-3, TIMP-4; MMP1, MMP2, MMP3, Batimastat (BB-94), TAPI-2,10-phenanthroline, and marimastat. The composition may contain anti-PDGF compound(s) such as imatinib mesylate (Gleevec®), sunitinib malate (Sutent®) which has anti-PDGF activity in addition to anti-VEGF activity, and/or anti-leukotriene(s) such as genleuton, montelukast, cinalukast, zafirlukast, praniukast, zileuton, BAYX1005, LY171883, and MK-571 to account for the involvement of factors besides VEGF in neovascularization. The composition may additionally contain other agents including, but not limited to, transforming growth factor β (TGFβ), interleukin-10 (IL-10), aspirin, a vitamin, and/or an antineoplastic agent.

Formulations may be prepared using a physiological saline solution as a vehicle. The pH of an ophthalmic formulation may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

The formulations may also contain pharmaceutically acceptable excipients known to one skilled in the art such as preservatives, stabilizers, surfactants, chelating agents, antioxidants such a vitamin C, etc. Preservatives include, but are not limited to, benzalkonium chloride, chlorobutanol, thimerosal, phenylmercuric acetate and phenylmercuric nitrate. A surfactant may be Tween 80. Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc. In one embodiment, the agent may be formulated in a controlled release system (i.e., delayed release formulations and/or extended release formulations) such as polylactic or polyglycolic acid, silicone, hema, and/or polycaprolactone microspheres, microcapsules, microparticles, nanospheres, nanocapsules, nanoparticles, etc.

In various embodiments, the compositions may contain other agents. The indications, effective doses, formulations, contraindications, vendors, etc. of these are available or are known to one skilled in the art. It will be appreciated that the agents include pharmaceutically acceptable salts and derivatives.

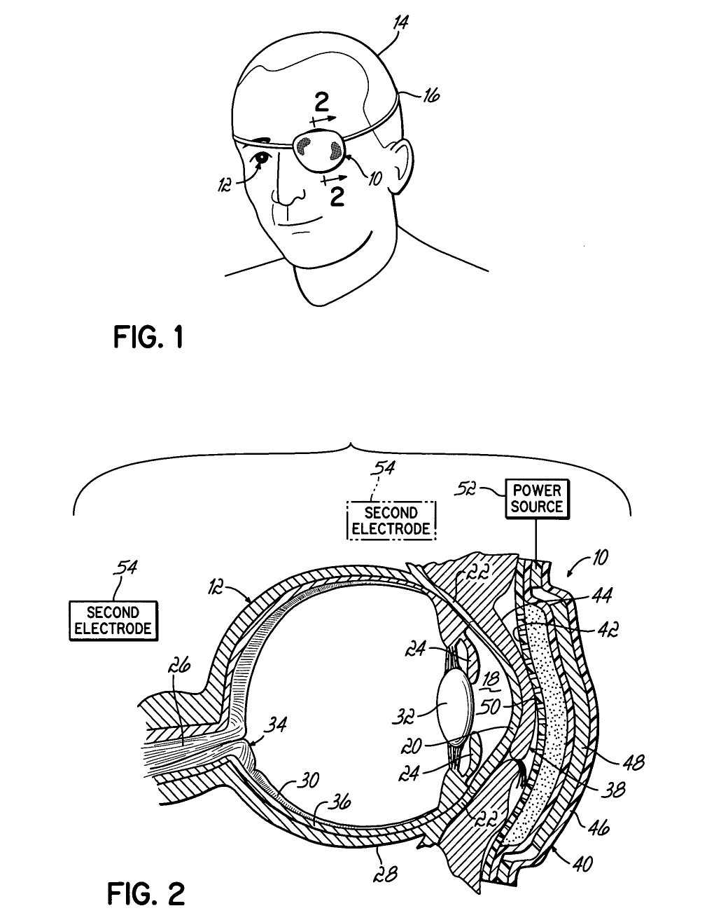

FIG. 1 is a perspective view of an agent delivery device 10 that facilitates administration of an agent into and/or throughout the eye 12 of a patient 14. Although FIG. 1 illustrates the patient 14 as being human those of ordinary skill in the art will recognize that embodiments of the invention may be used on other mammals. In one embodiment, the agent delivery device 10 is configured as an eye patch or eye cup that at least partially covers or overlies the eye 12. The device 10 may be secured to the patient 14 using a connecting member 16, such as an elastic band that may be resiliently stretched so as to position the band around the head of the patient and then released so as to secure the device 10 to the patient 14. Other types of connecting members may also be used with the invention. For example, hook and loop type of fasteners may be used to secure the device 10 to the patient 14. Alternatively, biocompatible adhesives may be used to secure the device 10 to the patient 14. Those of ordinary skill in the art will recognize a wide range of connecting members that may be used to secure the device 10 to the patient 14 so as to overlie the eye 12.

FIG. 2 is a schematic cross-sectional view of a mammilian eye 12 showing the anterior chamber 18, cornea 20, conjunctiva 22, iris 24, optic nerve 26, sclera 28, macula lutea 30, lens 32, retina 34 and choroid 36. The eye 12 further includes an eyelid 38 that overlies the cornea 20 when the eye 12 is closed. In one embodiment, the therapeutic agent is delivered to the eye 12 using electromotive drug administration, also referred to as iontophoresis, that is applied through the eyelid 38. The device 10 may be positioned proximate the eye 12 to facilitate iontophoretic administration of the agent.

Device 10 includes a housing body 40 having an inner surface 42 adapted to contact at least a portion of the outer surface 44 of the eyelid 38, and an outer surface 46 opposite the inner surface 42 that faces away from the eye 12. The device 10 may generally have any shape, e.g., circular, oval, square, or any other shape that effectively covers the eye 12 or at least makes sufficient contact with the eyelid 38. The device 10 includes a first electrode 48 in housing 40, i.e., an anode and/or cathode depending upon the charge state of the agent being delivered. The first electrode 48 is electrically insulated from outer surface 46 but is in electrical communication with at least a conductive portion 50 of inner surface 42. In this way, for example, electric current from the first electrode 48 cannot flow to outer surface 46 but may flow to conductive portion 50 of the inner surface 42. This allows a patient to touch the outer surface of the device 10 and possibly a portion of inner surface 42 without risk of electric shock, while current is permitted to flow into the eye 12 through conductive portion 50 of inner surface 42 and through the eyelid 38, as explained in more detail below. An electrically conducting gel, cream, lubricant, etc. may be applied to at least one of the eyelid or the inner surface 42 of the device 10 to enhance the electrical connection between the device 10 and the eyelid 38. The device 10 is also operatively coupled to a power source, schematically shown at 52, for supplying power to first electrode 48. In one embodiment, device 10 may include a battery (not shown) for supplying power to first electrode 48. The battery may be disposable or rechargeable and may be carried by housing 40 so as to be easily accessible through, for example, the outer surface 46 of device 10. The invention, however, is not so limited as other power sources, including external power sources, may be used to supply power to first electrode 48.

The device 10 may include a second electrode of opposite polarity (cathode and/or anode) shown schematically at 54, positioned at a site spaced from the first electrode 48 so as to define an electrically conductive path between the two electrodes 48, 54 and through the eye 12. By way of example, the second electrode 54 may be positioned within the body, such as behind the eye 12. Alternately, second electrode 54 may be positioned outside the body of the patient. In one embodiment, electrode 54 may be positioned behind the patient's head, on the patient's face, mouth, or forehead, or on other structures around the eye 12, illustrated in phantom in FIG. 2. Those of ordinary skill in the art will recognize the appropriate location of second electrode 54, depending on the position of the first electrode 48 so as to ensure delivery of the agent to a selective portion or structure of the eye 12 using iontophoresis.

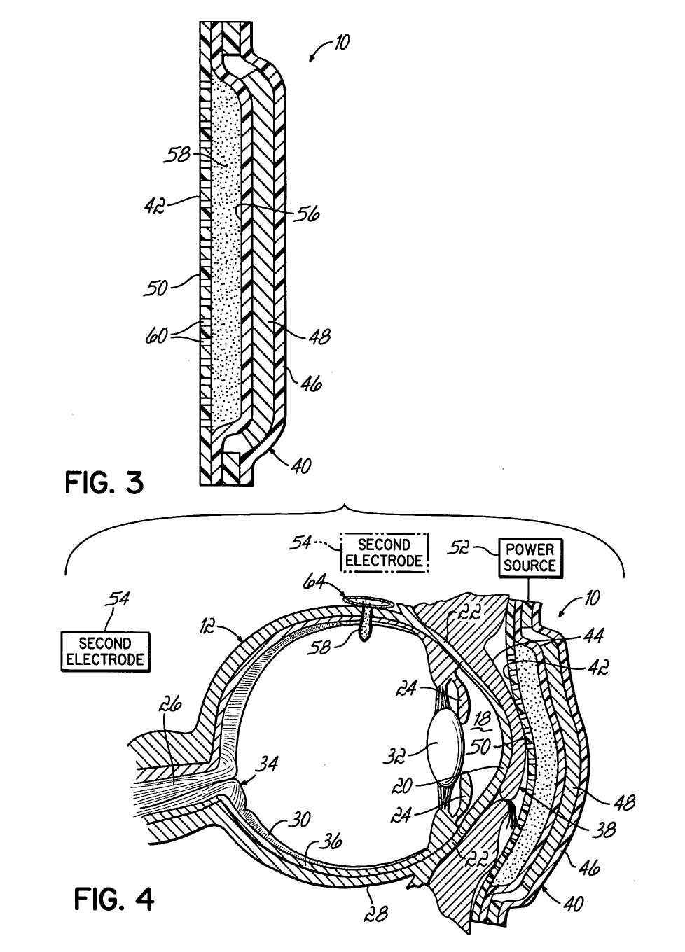

An agent may be introduced into the eye 12 in several ways and then disseminated throughout the eye 12 using the iontophoretic device 10. For instance, the agent may be introduced through topical administration or provided from a depot. The depot may be implanted inside the iontophoresis device or may be implanted under the skin, under the conjunctiva, under the sclera, or another location inside the eye. Electrical discharge activates release of the agent from the depot, regardless of depot location. In one embodiment, as shown in FIG. 3, device 10 may itself include a reservoir 56 adapted to hold an agent 58 suitable for iontophoresis, i.e., is capable of being charged. Reservoir 56 is in fluid communication with conductive portion 50 of inner surface 42 so as to permit the agent 58 to diffuse or otherwise be transported through inner surface 42 and into the eye 12 through eyelid 38. In this way, at least a portion of inner surface 42 operates as a diffusible barrier that allows the agent 58 to move from the reservoir 56 and into the eye 12. In essence, inner surface 42 facilitates control of the rate at which agent 58 moves into the eye 12. For example, inner surface 42 may include at least one opening or aperture 60 that permits fluid communication between the reservoir 56 and the eye 12. The aperture(s) 60 may have a wide variety of sizes and configurations depending on the preferences or requirements of a particular application. For example, the aperture(s) 60 may be one or more perforations, fenestrations, holes, slits, and/or slots, and other configurations known in the art. The shape of the aperture(s) 60 may also vary and may be circular, square, rectangular, elliptical, etc. or combinations of shapes. By way of example, FIG. 3 shows a device 10 where aperture(s) 60 are configured as circular holes. The size of aperture(s) 60 may be selected depending on the preferences or requirements of a particular application. For example, the aperture(s) 60 may have an identifiable cross dimension (such as diameter, slot length, etc.) that ranges from a few gm up to several mm (e.g., 10 mm). The size of aperture(s) 60 may vary from device to device, and may also vary on the same device. In one embodiment, the device 10 may have walls or other types of closures that selectively reduce or prevent the release of agent 58. The closures may reduce the size of aperture(s) 60 or alternately, completely close aperture(s) 60.

In operation, the device 10 is positioned on the head of the patient 14 so as to overlie the eye 12 that is being treated (see FIG. 1). The first electrode 48 is self or non-self activated using power source 52 causing a flow of current between the two electrodes 48, 54 and through the eye 12. For instance, the patient or the patient's caregiver may activate the device, or the device may be activated remotely by, for example, a physician. When current is applied, an electrical potential difference is generated that facilitates movement of agent 58 out of reservoir 56, through inner surface 42, into and through eyelid 38 and into the eye 12. Depending on the position of the second electrode 54, the agent 58 may be selectively delivered to the various structures of the eye 12, including the optic nerve 26, lens 32, retina 34, choroid 36, and other ocular structures such as the cornea 20, sclera 28, and eyelid 38 itself. For example, the device 10 may be used to treat diseases of the eyelid 38 by deliverying agents, including antibiotics, macrologies, NSAIDS, antivirals, anticancer drugs, etc., thereto. Due to electrical resistance, the device 10 generates heat that may be used to warm the eyelid 38 so as to facilitate secretions of the mibomian gland. The dose of agent 58 delivered to the eye 12 depends on the current and duration selected. For instance, the current may range between between 0.5 mA to about 4 mA. Those of ordinary skill in the art will recognize that the current may be greater than or less than these values depending on the particular application. Moreover, the treatment may be applied for anywhere between a few seconds to about 20 minutes. Again, however, those of ordinary skill in the art will recognize that the time duration may be greater or less than these values depending on the particular application. Those of ordinary skill in the art will recognize that the current and/or time duration may be manipulated so as to deliver the agent 58 into selective portions or structures of the eye 12. For example, the longer the time duration, the deeper within the eye 12 agent 58 is capable of penetrating.

Iontophoresis itself has no side effects and there is no pain associated with drug administration using this methodology. Moreover, the embodiment shown and described above is relatively non-invasive. Consequently, the device 10 may be used to treat various ocular diseases in a simplified manner that does not necessarily require a trip to the doctor's office or the expertise of a health care professional for its administration. Thus, patients themselves or those that care for the patient may administer agents to their eye(s) in their own home in accordance with an appropriate treatment plan. A medical practitioner need not be present. The patient can self administer the method. Even the treatment of transcorneal and transconjuntival conditions, which previously required a medical practitioner because of pain and or corneal abrasion with potential for corneal ulcer, infection, loss of sight, or loss of eye, can be safely treated by self-administration.

The reservoir 56 may be loaded with agent 58 in several ways. For example, in one embodiment, the reservoir 56 of device 10 may come pre-loaded with a specific agent or agents for the treatment of a particular ocular disease. In another embodiment, the reservoir 56 may be loaded with agent after the reservoir 56 has been inserted in device 10. For instance device 10 may permit resealable penetration by a needle or other conduit to fill/refill the reservoir 56 with an agent without removing the reservoir 56 from the device 10. In yet another embodiment, the reservoir 56 may be removable from device 10 such that if a different agent is to be administered to the eye 12 or if the reservoir 56 is empty and addition agent is desired, the old reservoir may be removed from device 10 and a new reservoir installed for continued treatment of the eye 12. In another embodiment, the reservoir 56 may include multiple chambers to contain multiple agents in segregated compartments using appropriate dividing walls. In this way, multiple agents may be delivered to the eye 12 using device 10.

Although the above embodiments introduce agent 58 into the eye 12 using device 10 itself, as noted above, the agent 58 may be introduced into the eye 12 in other ways. For example, the agent 58 may be introduced into the eye 12 by topical administration. The agent 58 may be formulated as a suspension, emulsion, gel, ointment, cream, lotion, eye drops, eye wash solutions, contact lens solutions, artificial tears, ophthalmic lubricants, and other ocular solutions suitable for topical administration to the eye. In this embodiment, the agent 58 may be topically administered on the cornea 20, conjunctiva 22, on the mucosal surface of the eyelid 38, or on the outer surface of the eyelid 38. For instance, in one embodiment, the electrically conductive layer on the eyelid 38 or inner surface 42 may include an agent for administration to the eye 12. Administration of agents 58 for treatment of diseases of other structures of the eye 12, such as the choroid, retina, and uvea, via local administration was previously restricted to systemic or invasive routes because it was thought that the higher concentrations of these agents in internal ocular structures required for efficacy could not be achieved by topical administration. However, an efficacious therapeutic concentration of a topically-administered agent in an ocular structure may be achieved by topically administering a supertherapeutic concentration for a duration such that a therapeutic concentration is attained in the diseased structure. Using iontophoresis to facilitate transport of the agent into the ocular structures allows a lower concentration of the agent to be used during topical administration but still achieve a therapeutic dose at the desired ocular structure.

While not bound by any theory, one reason this therapeutic concentration may be achieved with topical administration is that the structural affinity for lipids results in their accumulation in lipophilic regions of the choroid, retina, etc. Such topically administered agents can thus be used to treat pathologies that affect these structures without invasive methods, such as intraocular injection or systemic administration. Examples of pathologies include, but are not limited to, retinopathy including diabetic retinopathy, retinitis pigmentosa, age related macular degeneration, scleritis, uveitis, vasculitis, and oncological diseases affecting the eye such as retinoblastoma, choroidal melanoma, pre-malignant and malignant conjunctival melanoma. Such treatment may augment or enhance the effects of specific radiation treatments and/or chemotherapeutic agents. For example, macrolide and/or mycophenolic acid may be added in polymer form providing extended release to carboplatin, cisplatin, methotrexate, etc., in topical chemotherapy eye drops. Diseases such as diabetic retinopathy, retinitis pigmentosa, and age related macular degeneration are typically chronic so that treatment is prolonged, while diseases such as scleritis, uveitis and vasculitis may be acute with treatment occurring for a shorter duration, that is, over the course of the disease. The invention encompasses both types of treatment, as will subsequently be described.

The topically administered composition must cross ocular structures such as the conjunctiva and sclera to reach structures such as the choroid, retina, and uvea. In transit of the composition, a natural gradient of the active agent(s) may form within the eye. A structure such as the sclera may act as a depot or repository for the active agent(s), providing extended release. Thus, topical administration may provide results similar to a slow release formulation, as will be described. Such formulations desirably decrease the frequency of administration or dosing. For example, patients being treated for an ocular disease may have decreased visual acuity, and topical ocular administration of drugs may be difficult and/or uncomfortable for them. Reducing the frequency of administration enhances compliance, while providing a therapeutic dosage of the composition.

In this embodiment, once the agent 58 has been introduced into the eye 12, for example using topical administration, the device 10 is positioned on the head of the patient 14 so as to overlie the eye 12 that is being treated. The first electrode 48 is self or non-self activated using power source 52 causing a flow of current between the two electrodes 48, 54 and through the eye 12. When current is applied, an electrical potential difference is generated that facilitates movement of agent 58 away from the first electrode 48 and toward the second electrode 58 through the eye 12. In this embodiment, the device 10 does not require a reservoir 56 for introduction of the agent 58. As those of ordinary skill in the art will recognize, however, the same agent or another agent my be loaded into the reservoir 56 of device 10 and released into the eye 12 while simultaneously transporting the agent introduced via topical administration through the eye 12 as well. Thus, introduction of the agent into the eye 12 may occur via different routes (e.g., topical administration and through the device 10) substantially simultaneously. Alternately, the agent introduced by topical administration may be subject to iontopheresis prior to introducing an agent from device 10, or vice versa.

The device 10 may be used to facilitate movement of an agent through the eye that is introduced into the eye 12 by still another route. In one embodiment, and as shown in FIG. 4, agent 58 may be released from a device 64 that is located within the eye 12 itself and operates as a reservoir or depot for agent 58. Those of ordinary skill in the art will recognize such depot device. For example, such a reservoir device is disclosed in U.S. application Ser. No. 11/423,458, filed Apr. 4, 2005 and entitled “OCULAR DRUG DELIVERY”; and U.S. application Ser. No. 11/348,151, filed Feb. 6, 2006 and entitled “DEVICE FOR DELIVERY OF AN AGENT TO THE EYE AND OTHER SITES,” the latter disclosure of which is incorporated by reference herein in its entirety. The device 64 may be implanted through a minimally invasive surgical procedure that may be performed in a physician's office or on an outpatient bases. An anesthetic is administered to the patient (e.g., topical, local, etc.) as known to one of skilled in the art. A relatively small incision (about 5 mm) is made in the peribulbar conjunctiva 22 such that a pocket is created between the conjuctiva 22 and the sclera 28. The device 64 may be implanted in the pocket for release of the agent 58 into the sclera 28 or the vitreous cavity. The device may be secured within the eye 12 by, for example, one or more sutures, a biocompatible sealant, adhesive, etc. The device 64 may introduce the agent through a diffusion process or other process known to those of ordinary skill in the art for introducing the agent 58 from device 64. For instance, the device 64 may be configured so that release from the device 64 may be regulated remotely, as more fully disclosed in the U.S. patent applications noted above.

In this embodiment, once the agent 58 has been introduced into the eye 12 using agent depot 64, the device 10 is positioned on the head of the patient 14 so as to overlie the eye 12 that is being treated. The first electrode 48 is self or non-self activated using power source 52 causing a flow of current between the two electrodes 48, 54 and through the eye 12. When current is applied, an electrical potential difference is generated that facilitates movement of agent 58 introduced from depot 64 away from the first electrode 48 and toward the second electrode 58 through the eye 12. This mode or route of introducing an agent into the eye 12 may be used alone or in combination with the other routes of agent introduction described above (i.e., topical administration and from device 10). As recognized by those of ordinary skill in the art, the introduction of an agent into the eye by the various routes may occur essentially simultaneously or a different times that may or may not overlap one another. Those of ordinary skill in the art will further recognize additional routes of introducing agent 58 into the eye 12 than those described above. For instance, the agent may be introduced into the eye through intraocular injection. This and other methods of introducing an agent into the eye known to those having skill in the art are contemplated to be within the scope of the invention.

In one embodiment, device 10 may be fabricated to be externally regulated. For example, dosing through the inner surface 42 and operation of the electrodes 48, 54 may be controlled by a software program that communicates with a microchip associated with the device 10. The program may be accessed, verified, altered, monitored, etc., even from a remote location. In embodiments, the release of agent 58 from the device 10 and/or activation of the electrodes 48, 54 may be pre-set, or may be manually regulated at the point of use, or may be regulated from a remote location. This may include volume, duration, rate, release intervals, etc. In one embodiment, the release of agent 58 is remotely controlled by electric stimulation. For example, the aperture(s) 60 may be partially or completely associated with a piezoelectric film, an electric erosion barrier, etc. Upon electric stimulation, the film or barrier is disrupted sufficiently to allow at least a portion of agent 58 in reservoir 56 to egress through the aperture(s) 60. If more than one aperture 60 is present, each aperture 60 may be associated with a film, barrier, etc. that requires different stimulation levels to disrupt, allowing selective control of the delivery of agent 58. The film or barrier may cover all or part of the aperture(s) 60, or be located adjacent an aperture(s) 60, in its association with the device 10. In another embodiment, the release of agent 58 through inner surface 42 is remotely controlled by microactivation, whereby the patient or device is fitted with a receiving device such as an antenna, and a radiofrequency identification (RF-ID) chip carrying a microactivator for causing the release of agent 58. An RF-ID interrogator is used to interrogate the receiving device, for example, from a remote location, providing power to the RF-ID chip and causing the RF-ID chip to trigger the microactivator by delivering an appropriate coded instruction to the RF-ID chip via radiofrequency signals.

Radio frequency (RF) telemetry may be used to remotely activate the device to release agent 58 through the inner surface 42 or remotely activate the electrodes 48, 54, as known to one skilled in the art. The circuitry, programming, and other components and their implementation are described in, e.g. U.S. Pat. No. 5,170,801 where a circuit in a capsule device receives RF signals and causes drug release from openings in the device; U.S. Pat. No. 5,820,589 where RF telemetry is used to program and/or reprogram power and/or flow rate information to an implanted pump to release a drug, with the pump containing an antenna and circuitry to receive a signal transmitted by an external remote device placed over the skin of the patient; upon receiving a signal, the circuitry changes the operating parameters and the new settings remain in place until new programming instructions are received by RF signals or other non-invasive telemetry in the circuitry; U.S. Pat. No. 5,312,453 describing an external programmer device that transmits RF encoded signals to an implanted device using programming that allows remote selection of parameters and settings for the implanted device; and U.S. Pat. No. 6,824,561, disclosing a hand-held device using RF, infrared, acoustic pulsed, or magnetic activating means where a surgeon, physician, or patient holds the device over the implant site and activates the device to release agent(s). Each of these patents is expressly incorporated by reference herein in its entirety.

These and other embodiments can be adapted by one skilled in the art. As described, the remote activating device may contain a microprocessor and at least one antenna to transmit RF signals to the implanted device. A programming circuit in the implanted device may contain at least one antenna to receive transmitted signals from the remote device and, upon detection of a signal, the programming circuit may cause release of agent 58 from an aperture(s) 60 and/or the activation of electrodes 48, 54. As a result, a, physician is able to remotely activate the device to release the agent 58 or initiate iontophoresis. Additional safety precautions may also be incorporated by one skilled in the art. As one example, the programming circuitry may be configured to respond only to a specific RF signal in order to avoid accidental activation of the device. As another example, the programming circuitry may be configured to incorporate pre-determined dosage information into the remote device in order to prevent remote activation of the device after a maximum dosage has been already released or a maximum duration time has been reached.

RF signals or other telemetry may also serve as a power supply for the device, circuit, and/or any other components. Thus, while operating the remote device, power may be transmitted to the device via the transmitted RF signal, and release of agent 58 or activation of electrodes 48, 54 may cease when the individual operating the remote device causes it to stop transmitting a signal (i.e., removing the power supply). Various modifications may be made to the embodiments above as known to one skilled in the art.

It should be understood that the embodiments shown and described in the specification are only preferred embodiments of the inventor who is skilled in the art and are not limiting in any way. Therefore, various changes, modifications or alterations to these embodiments may be made or resorted to without departing from the spirit of the invention and the scope of the following claims.

What is claimed is:

1. An apparatus for delivering an agent to the eye using iontophoresis through the eyelid of a: patient, comprising:
a housing having an inner surface adapted to be in electrical communication with the eyelid and an outer surface opposite the inner surface;
a first electrode positioned in the housing and in electrical communication with the inner surface of the housing.

2. The apparatus of claim 1, further comprising:
a power source in electrical communication with the first electrode.

3. The apparatus of claim 2, wherein the power source is a battery carried by the housing.

4. The apparatus of claim 2, further comprising:
a second electrode in electrical communication with the patient proximate the eye, the second electrode adapted to cooperate with the first electrode such that current passes between the first and second electrodes and through the eye when the first electrode is in electrical communication with the power source.

5. The apparatus of claim 1, further comprising:
a reservoir in the housing adapted to hold at least one agent and in fluid communication with the inner surface of the housing, the inner surface of the housing operating as an adjustable barrier and having at least one aperture for permitting fluid communication between the reservoir and the eyelid so as to introduce the agent into the eye and through the eyelid.

6. The apparatus of claim 5, wherein the reservoir includes two or more compartments.

7. The apparatus of claim 1, wherein the housing is configured as an eye patch.

8. The apparatus of claim 7, further comprising:
a connecting member adapted to secure the housing to the eye of the patient.

9. A method of making a drug delivery device for delivering an agent to the eye using iontophoresis through the eyelid of a patient, comprising:
providing a housing having an inner surface adapted to be in electrical communication with the eyelid of the patient;
disposing a first electrode in the housing so as to be in electrical communication with the inner surface of the housing.

10. The method of claim 9, further comprising:
providing a power source in electrical communication with the first electrode.

11. The method of claim 10, further comprising:
providing a second electrode adapted to be placed in electrical communication with the patient proximate the eye, the second electrode adapted to cooperate with the first electrode such that current passes between the first and second electrodes and through the eye when the first electrode is in electrical communication with the power source.

12. The method of claim 9, further comprising:
disposing a reservoir in the housing adapted to hold at least one agent and in fluid communication with the inner surface of the housing, the inner surface of the housing operating as an adjustable barrier and having at least one aperture for permitting fluid communication between the reservoir and the eyelid so as to introduce the agent into the eye and through the eyelid.

13. A method for ocular drug delivery, comprising:
introducing an agent into an eye of a patient; and
selectively directing the agent through the eye using iontophoresis applied through an eyelid of the patient.

14. The method of claim 13, wherein introducing an agent into the eye further comprises:
introducing the agent into the eye though topical administration.

15. The method of claim 14, wherein the agent is selected from the group consisting of a suspension, emulsion, gel, ointment, cream, lotion, eye drops, eye wash, contact lens solution, artificial tears, ophthalmic lubricants, and combinations thereof.

16. The method of claim 14, wherein the agent is introduced onto at least one of the cornea, conjunctiva, and eyelid.

17. The method of claim 13, wherein introducing an agent into the eye further comprises:
introducing the agent into the eye through an agent depot internal to the eye, the agent depot adapted to release agent from the depot and into the eye.

18. The method of claim 17, wherein the release of agent from the agent depot is regulated remotely.

19. The method of claim 13, wherein introducing an agent into the eye further comprises:
introducing the agent into the eye through a reservoir external to the eye.

20. The method of claim 19, wherein the reservoir is in fluid communication with the eyelid so the agent is introduced into the eye through the eyelid.

21. The method of claim 19, wherein the release of agent from the reservoir is regulated remotely.

22. The method of claim 19, wherein the reservoir is separated into at least two compartments each containing an agent, the release of agent from each of the compartments being independently controlled.

23. The method of claim 13, wherein directing the agent through the eye using iontophoresis further comprises:
positioning a first electrode in electrical communication with the eyelid;
positioning a second electrode in relation to the first electrode to facilitate movement of the agent into a selective structure of the eye; and
energizing at least one of the electrodes to cause current to flow between the electrodes and through at least a portion of the eye.

24. The method of claim 23, wherein the second electrode is located at one of behind the head, on the face, on the mouth, and on the forehead.

25. The method of claim 23 further comprising:
varying at least one of current magnitude or current duration to control agent delivery to a selective ocular structure.

26. The method of claim 23, wherein energizing the at least one of the electrodes is regulated remotely.

27. The method of claim 13, wherein the agent is selected from the group consisting of an antibiotic, anti-inflammatory, anti-proliferative, hormone, cytokine, growth factor, antibody, immunie modulator, vector for gene therapy, oligonucleotide, enzyme, enzyme inhibitors, and combinations thereof.

28. The method of claim 13 wherein the drug is in a nanotechnology formulation.
